(19) United States
(12) Patent Application Publication
(10) Pub. No.: US 2004/0142572 A1
(43) Pub. Date: Jul. 22, 2004

(54) Apparatus and method for selectively inducing hydrophobicity in a single barrel of a multibarrel ion selective microelectrode

(76) Inventors: Jason S. T. Deveau, Guelph (CA); Michael I. Lindinger, Guelph (CA)

Correspondence Address: DOWELL & DOWELL PC, SUITE 309, 1215 JEFFERSON DAVIS HIGHWAY, ARLINGTON, VA, 22202

(21) Appl. No.: 10/345,409

(22) Filed: Jan. 16, 2003

(51) Int. Cl. A61L 2/00; H01L 21/311; B05D 3/00; C23C 16/00

(52) U.S. Cl. 438/694

(57) ABSTRACT

The present invention provides an apparatus and method for inducing hydrophobicity in selected barrels of multi-barreled microelectrodes made from glass microcapillaries in a consistent and simultaneous fashion. The apparatus for producing hydrophobicity under substantially identical conditions includes a housing defining a gas-tight chamber and made of a material substantially inert in the presence of a volatile hydrophobicity-inducing solution. The housing includes a port in communication with the chamber for injection of a volatile hydrophobicity-inducing solution into the chamber and the housing also includes a plurality of ports in communication with the chamber for receiving a single or multibarreled microelectrode therein, each of the ports having a seal for forming a gas-tight seal around each microcapillary in the port.

### FIELD OF THE INVENTION

The present invention relates generally to an apparatus and method for consistently and simultaneously inducing hydrophobicity in selected barrels of multibarreled microelectrodes.

### BACKGROUND OF THE INVENTION

Microelectrodes are used to measure electrical potentials and ion activities inside the cellular or intercellular space of organic tissue (e.g animals and plants) in biological and medical research. Microelectrodes have been in use for about 80 years and possibly longer, and have typically been made from very fine glass microcapillaries filled with saline solution. The extension of the use of microelectrodes to include combination ion-selective microelectrodes (e.g. pH microelectrodes) and reference electrodes has developed over the past 30 years. The basic principles of the measurement of ionic activity were developed over 100 years ago by Nernst whose equation, with refinements, is relied upon in the measurements.

The microelectrodes are formed from glass microcapillaries that are typically heated and drawn down to less than 1 micron diameter (but also may be drawn down to 0.1 micron. See Gregor R., Weidtke C., Schlatter E., Wittner M. and Gebler B. (1984a) K⁺ activity of cells of isolated perfused cortical thick ascending limbs of rabbit kidney. Pflügers Arch. 401, 52). In some configurations two or more glass microcapillaries are drawn down simultaneously to form a multibarrel structure so that microelectrodes with different functions can be brought to the same cellular or intercellular space being investigated.

Ion-selective liquid resins can be made or purchased commercially (e.g. Fluka Chemical Corp., Ronkonkoma, N.Y., USA) for sensing a wide range of ions. These can be introduced as a small plug (herein referred to as a membrane) in the end of a single microcapillary barrel of a multibarrel assembly, thereby forming the ion-selective microelectrode. The resin membrane adheres to the inside of the microcapillary provided the glass has been rendered hydrophobic by treatment with a hydrophobicity-inducing compound (e.g. a silane compound such as dimethyldicholorosilane or tributylchlorosilane). Historically, this was achieved by immersing the glass microcapillary in a dilute solution of silane-in-chloroform, and then heating it to about 180° C. In a multi-barrel microelectrode it is important not to introduce the hydrophobicity-inducing solution to any barrel whose ends are not destined to be filled with the ion-selective resin. Those other barrels must remain hydrophilic.

The primary role of the hydrophobicity-inducing process is to ensure good bonding between the ion-selective resin and the microcapillary and to prevent changes in the integrity or location of the resin membrane. After the resin is introduced into the tip of the hydrophobic barrel, the rest of the barrel is filled with a conductive, aqueous saline solution.

The hydrophobicity-inducing process is very problematic from several perspectives. Many hydrophobicity-inducing substances (e.g. silane) are very volatile and extremely toxic. Said substances must be restricted to entering only those barrels destined to become ion-selective. Since the microelectrodes are very fragile they must not physically contact anything during the hydrophobicity-inducing process. This can become difficult, as a great deal of physical manipulation is usually required. Various methods have been devised to attempt to overcome these difficulties, but they suffer from several drawbacks including inconsistent results, and success rates typically in the range of 10% to 75%. This is due to various factors including incomplete hydrophobicity, contamination by hydrophobicity-inducing compounds, or inconsistent tip morphology. All the methods used require multiple steps to accomplish the hydrophobicity-inducing process and some methods require the use of specialty glasses.

Virtually every lab that builds ion-selective multi-barreled microelectrodes utilizes a different method of construction. Although they meet with varied levels of success, they are all complicated multi-step procedures (Felle H., Bertl A. (1986) The fabrication of H⁺-selective liquid-membrane micro-electrodes for use in plant cells. Journal of Experimental Botany 37 (182): 1416-1428.; Benes V., Kilger C., Voss H., Paabo S., Ansorge W. (1997) Fabrication of ion-selective microelectrodes by a centrifugation/suction method. Biotechniques 23 (1): 100-101.; Semb S. O., Amundsen B., Sejersted O. M. (1997) A new improved way of making double-barreled ion-selective micro-electrodes. Acta Physiol Scand 161: 1-5.). A single unit for facilitating desiccation, selectively inducing hydrophobicity, and cooling batches of multi-barreled microelectrodes is a highly desirable and advantageous apparatus for many applications (e.g. electrophysiology).

A major difficulty with the use of multi-barreled microelectrodes has been to selectively induce hydrophobicity in only one barrel (Zeuthen, T. (1980) in E. Boulpaep, G. Giebisch (Eds.), How to make and use double-barrelled ion-selective microelectrodes, Current Topics in Membranes and Transport, vol. 13). The resultant condition facilitates the uptake and retention of the ion-selective resin, leaving the reference barrel hydrophilic and thus, resin-free. The apparatus 10 disclosed herein overcomes this difficulty. For example, some researchers use heat lamps and inject hydrophobicity-inducing solutions directly into the barrels, but this increases the risk of inadvertent silanization of several barrels, and is highly labor intensive (Walker D. J., Smith S. J., Miller. A J. (1995) Simultaneous measurement of intracellular pH and K⁺ or NO₃⁻ in barley root cells using triple-barreled ion-selective microelectrodes. Plant Physiology 108: 743-751).

Others have developed chamber-like apparatuses to isolate barrels for inducing hydrophobicity, but they must be manipulated during the baking process, can only treat one microelectrode at a time, require regular replacement because repeated use loosens the electrode-chamber seal, and often yield inconsistent results (Voipio, J. Pasternack, M. MacLeod, K. in D. Ogden (Ed.), Ion-sensitive microelectrodes, Microelectrode Techniques—The Plymouth Workshop Handbook 2^{nd} Ed., The Company of Biologists Ltd., Cambridge, 1994). Some designs require the manipulation of the microcapillaries prior to and following the induction of hydrophobicity, and require the experimenter to use theta-style tubing (Semb S. O., Amundsen B., Sejersted O. M. (1997) A new improved way of making double-barreled ion-selective micro-electrodes. Acta Physiol Scand 161: 1-5).

Theta glass is glass tubing with a thin septum of glass dividing it into two distinct longitudinal chambers. A limitation of this design is the likely development of microcracks in the septum during the pulling process, which results in electrical shorts between the reference, and ion-selective barrels (Brown, K. T. Flaming, D. G. in A. D. Smith (Ed.), Advanced Micropipette Techniques for Cell Physiology, IBRO Handbook Series: Methods in the Neurosciences, vol. 9, John Wiley & Sons, New York, 1995; Ammann, D. Ion-Selective Microelectrodes—Principles, Design and Application, Springer-Verlag, Berlin, Heidelberg, New York, Tokyo, 1986).

Therefore it would be most advantageous to provide a method and device for the selective induction of hydrophobicity to multi-barreled microelectrodes which facilitates desiccation, and cooling. Such a device would physically support microelectrodes during the induction of hydrophobicity and any subsequent solvent evaporation while requiring minimal manipulation by the operator producing the microelectrodes.

### SUMMARY OF THE INVENTION

An objective of the present invention is to provide an apparatus that produces a consistent, selective induction of hydrophobicity in multi-barreled microelectrodes while minimizing operator manipulation, and enhancing efficiency and consistency.

In one aspect of the invention there is provided an apparatus for producing selective hydrophobicity in single or multiple glass microcapillaries, comprising;

a housing defining an gas-tight chamber and made of a material substantially inert in the presence of a volatile hydrophobicity-inducing agent, the housing including a port in communication with said chamber for injection of a the volatile hydrophobicity-inducing agent into said chamber, said housing including at least two ports in communication with said chamber for receiving a microcapillary therein, each of said at least two ports having a seal for forming a gas-tight seal around each microcapillary in said port.

The present invention also provides an apparatus for producing selective hydrophobicity in batches of glass microelectrodes under substantially identical conditions, comprising;

a housing defining an gas-tight chamber and made of a material substantially inert in the presence of a volatile hydrophobicity-inducing agent, the housing including an inlet port in communication with said chamber for injection of the volatile hydrophobicity-inducing agent into said chamber, said housing including a plurality of ports in communication with said chamber for receiving a microcapillary therein, each of said at least two ports having a seal for forming a gas-tight seal around each microcapillary in said port.

In another aspect of the invention there is provided a method for producing selective hydrophobicity in batches of glass microcapillaries under substantially identical conditions, comprising the steps of;

providing a housing defining an gas-tight chamber and made of a material substantially inert in the presence of a volatile hydrophobicity-inducing agent, the housing including an inlet port in communication with said chamber for injection of a volatile hydrophobicity-inducing agent into said chamber, said housing including a plurality of ports in communication with said chamber for receiving a microcapillary therein, each of said at least two ports having a seal for forming a gas-tight seal around each microcapillary in said port;

inserting a microcapillary into each port of said plurality of ports and sealing each microcapillary therein;

injecting the hydrophobicity-inducing agent through the inlet port in the housing whereby the inner surfaces of the glass barrel of each microcapillary sealed in the ports is exposed to the volatile hydrophobicity-inducing agent thereby producing a batch of microcapillaries with inner surfaces of the glass barrel of each microcapillary being hydrophobic; and

removing each microcapillary from said housing.

### BRIEF DESCRIPTION OF THE DRAWINGS

Preferred embodiments of the invention will now be described, by way of example only, with reference to the drawings, in which:

FIG. 1 shows an apparatus for selectively inducing hydrophobicity in glass microelectrodes constructed in accordance with the present invention;

FIG. 2 shows an exploded view of the main body of the apparatus, with injection port and microelectrode ports (five shown) absent; and

FIG. 3 shows exploded views of both injection ports and microelectrode ports (five shown) forming part of the apparatus of FIGS. 1 and 2.

### DETAILED DESCRIPTION OF THE INVENTION

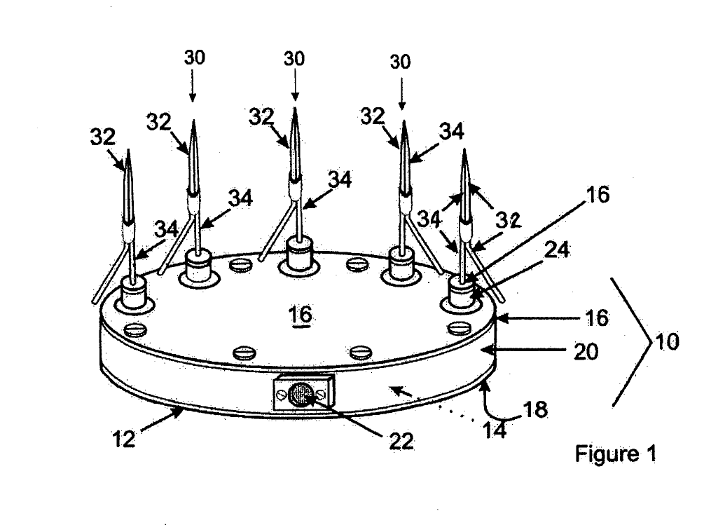

Referring to FIG. 1, a perspective view of an assembled multi-port apparatus for producing hydrophobicity in the glass barrels of microcapillaries is shown generally at 10. Apparatus 10 includes a housing 12 enclosing a chamber 14. Housing 12 includes a top plate 16, a bottom plate 18 and side wall 20. An injection port or septum 22 is located in the side wall 20 of housing 12 for coupling a gas supply to housing 12 which injects a volatile hydrophobic inducing fluid into chamber 14. The housing 12 as shown in FIG. 1 is cylindrical in shape but it will be appreciated that it may be any shape and is not restricted to being cylindrical. It is made of heat resistant polymers or metal. All seams preferably have heat resistant seals such as heat resistant Viton O-rings which can be easily replaced. Several ports 24 are located in the top plate 16 with each port 24 having a hole 26 having a size sufficient to receive a glass microcapillary 34 from a microelectrode bundle 30 therein with each port 24 including a heat resistant O-ring designed to seal around each glass microcapillary 34 when the microcapillary is inserted into the port.

FIG. 1 shows each port 24 having a multi-barrel ion-selective microelectrode bundle 30 (two microcapillaries 32 and 34 per bundle shown). The microcapillary 34 destined to become hydrophobic from each bundle 30 is inserted into a port 24 and the port fittings are then tightened to form a gas-tight seal. The chamber 14 with the microcapillaries 34 coupled thereto is then baked for about 1 hour at about 180° C. The appropriate volume of hydrophobicity inducing agent which is preferably a silane- and solvent-containing fluid (e.g 100 microlitres of 5% dimethyldichlorosilane in toluene) is injected directly into chamber 14 through port 22. In this example, the immediate vaporization of the silane-containing gas creates a positive pressure thereby forcing silane into the exposed glass barrel of each microcapillary 34. In this example, the microelectrodes 34 and the device continue to bake for approximately one more hour, and as the solvent dissipates, the silane adheres to the inside surface of the exposed barrel of each exposed microcapillary 34. While silane is preferred for producing hydrophobicity, it will be appreciated that any fluid that induces hydrophobicity can be used.

Apparatus 10 allows for the mounting of several multi-barrel microelectrodes at once for batch hydrophobicity-induction through the series of ports 24 in the housing 12. The protocol is as follows: microcapillaries 30 are initially dried (while mounted in the housing 12 prior to the injection of hydrophobicity-inducing solution) in an oven. Alternatively, apparatus 10 may include a heater directly built into the housing to heat the housing and microelectrodes.

Once the microelectrodes are fully desiccated, an injection of hydrophobicity-inducing solution through septum port 22 in the housing 12 effectively exposes only the microcapillary destined to become ion-selective of each multi-barreled microelectrode, which is sealed in the ports 24 to the hydrophobicity-inducing solution (this does not require any manipulation of the microelectrodes). The rapidly expanding solvent in the solution forces the hydrophobicity-inducing chemical into only the exposed barrels 34, and has no access to adjacent barrel(s) 32 on the same microcapillary bundle 30. The microelectrodes remain in the oven while the barrels silanize and the solvent evaporates. The apparatus 10 is then removed to a desiccator during cooling. Subsequently, the microelectrodes 30 are removed to a desiccator for 12 hours to 30 days prior to use. Apparatus 10 facilitates the simultaneous production of several, high quality ion-selective microelectrodes from hydrophobic microcapillaries and the inventors have noted a success rate approaching 90%. There are fewer steps in the protocol and less manipulation of the microelectrodes, which results in less chance of breakage and improved, consistent results.

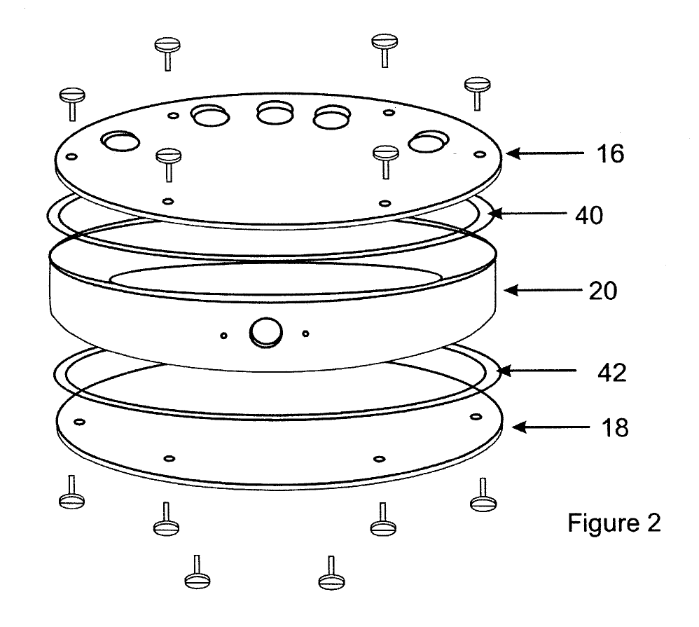

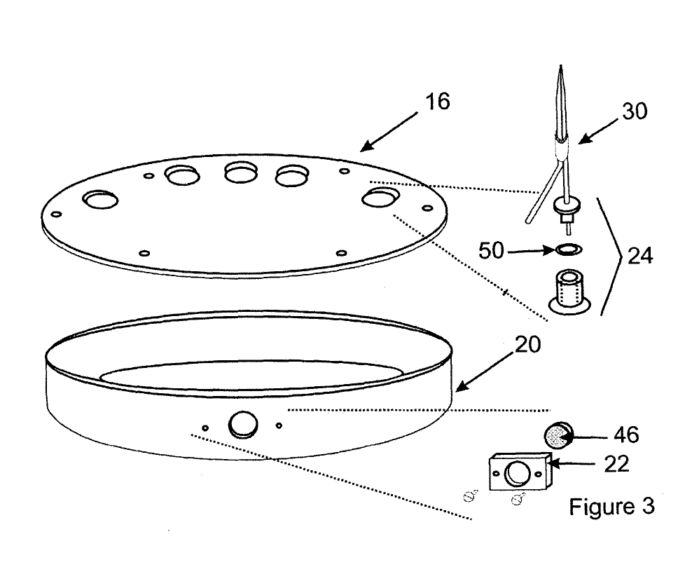

FIG. 2 shows the exploded view of the housing 12 showing the gas tight O-rings 40 and 42 for sealing top plate 16 and bottom plate 18 to wall 20 respectively. FIG. 3 shows exploded injection port 22 with gas tight injection septum 46 and one sample microcapillary port fitting 24 with gas tight ring 50 and sample double-barreled microelectrode 30.

There are many benefits to using multi-barreled ion-selective microelectrodes over a battery of single electrodes. First, the location of an intracellular microelectrode tip is of great importance to the experimenter; specifically, the tip of the reference electrode should be proximal to the ion-selective tip to ensure they are measuring the same cellular or intercellular space. The fused tip design of the multi-barreled microelectrode makes relocation easy and reliable within cellular or intercellular space. (Ammann, D. Ion-Selective Microelectrodes—Principles, Design and Application, Springer-Verlag, Berlin, Heidelberg, New York, Tokyo, 1986).

Second, fewer micromanipulators are required, which translates into less money, less effort, and less time involved in the set-up of an experiment. The electrodes can be used for the injection or current, dyes, or chemicals.

Apparatus 10 disclosed herein allows the multi-step process of drying, baking and producing hydrophobicity in the glass barrels of microelectrodes to be conducted with less manipulation and greatly reduced possibility of hydrophobicity-inducing solution contamination. In addition, it allows the use of any variety of glass tubing (e.g. microcapillaries), which is compatible with many brands of conventional half-cell/holders (e.g. World Precision Instruments). The housing 12 is therefore very useful for facilitating the selective hydrophobicity of microelectrodes for the study of cellular or intercellular space of organic tissue (e.g. animals and plants) or solutions in biological and medical research. It allows one to selectively induce hydrophobicity in only the barrel(s) desired, leaving the reference barrel uncontaminated and permits multiple microelectrodes to be treated at the same time, limited only by the number of ports in housing 12. Therefore, the present apparatus economizes and standardizes the use of hydrophobicity-inducing agents for consistent reproduction. It further reduces the time and effort required for manufacture of ion-selective multi-barreled microelectrodes.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. An apparatus for producing hydrophobicity in glass microcapillaries, comprising;
a housing defining an gas-tight chamber and made of a material substantially inert in the presence of a volatile hydrophobicity-inducing agent, the housing including a port in communication with said chamber for injection of a the volatile hydrophobicity-inducing agent into said chamber, said housing including at least two ports in communication with said chamber for receiving a microcapillary therein, each of said at least two ports having a seal for forming a gas-tight seal around each microcapillary in said port.

2. The apparatus according to claim 1 including a heat resistant housing with heat-resistant seals and fittings that undergo minimal deterioration.

3. An apparatus for producing selective hydrophobicity in batches of glass microelectrodes under substantially identical conditions, comprising;
a housing defining an gas-tight chamber and made of a material substantially inert in the presence of a volatile hydrophobicity-inducing agent, the housing including an inlet port in communication with said chamber for injection of the volatile hydrophobicity-inducing agent into said chamber, said housing including a plurality of ports in communication with said chamber for receiving a microcapillary therein, each of said at least two ports having a seal for forming a gas-tight seal around each microcapillary in said port.

4. The apparatus according to claim 3 including a heating means for heating said housing.

5. A method for producing selective hydrophobicity in batches of glass microcapillaries under substantially identical conditions, comprising the steps of;
providing a housing defining an gas-tight chamber and made of a material substantially inert in the presence of a volatile hydrophobicity-inducing agent, the housing including an inlet port in communication with said chamber for injection of a volatile hydrophobicity-inducing agent into said chamber, said housing including a plurality of ports in communication with said chamber for receiving a microcapillary therein, each of said at least two ports having a seal for forming a gas-tight seal around each microcapillary in said port;
inserting a microcapillary into each port of said plurality of ports and sealing each microcapillary therein;
injecting the hydrophobicity-inducing agent through the inlet port in the housing whereby the inner surfaces of the glass barrel of each microcapillary sealed in the ports is exposed to the volatile hydrophobicity-inducing agent thereby producing a batch of microcapillaries with inner surfaces of the glass barrel of each microcapillary being hydrophobic; and
removing each microcapillary from said housing.

6. The method according to claim 5 including a step of heating the housing and each microcapillary coupled to said housing for a pre-selected length of time and thereafter cooling said housing prior to removing each microcapillary.

7. The method according to claim 5 wherein the volatile hydrophobicity-inducing fluid can be 5% dimethyldichlorosilane in toluene.

8. The method according to claim 5 wherein after the step of inserting a microcapillary into each port of said plurality of ports and sealing each microcapillary therein, heating the housing and microcapillary to a temperature of about 180° C. for about one hour prior to injection, and maintaining this temperature for about one hour following the injection of the hydrophobicity-inducing fluid.
